(19) United States
(12) Patent Application Publication
(10) Pub. No.: US 2026/0345628 A1
(43) Pub. Date: Nov. 5, 2026

(54) Compounds and Methods for Treating Neurodegenerative Disease, Atherosclerosls and Insulin-resitant Diabetes

(71) Applicant: Donald L. Barbeau, Savannah, GA (US)

(72) Inventor: Donald L. Barbeau, Savannah, GA (US)

(21) Appl. No.: 18/831,563

(22) Filed: Apr. 23, 2025

(51) Int. Cl. A61K 31/5415

(52) U.S. Cl. CPC A61K 31/5415

(57) ABSTRACT

The present invention relates to compounds and pharmaceutical compositions for treating a subject having an intractable non-cancer in need of therapy thereof comprising administering to the subject a compound in an amount effective in inhibiting progression of the intractable disease, said compound having the formula:

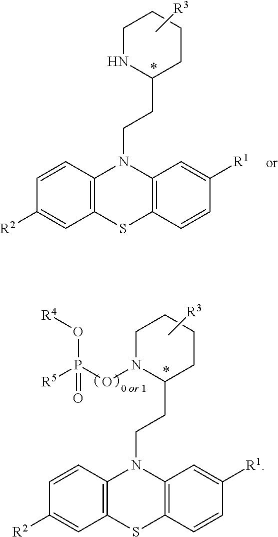

### BACKGROUND OF THE INVENTION

The cytoskeleton plays an important role in regulating the cellular morphology, cellular cytoplasmic signaling, cellular migration and resistance to disease therapies through regulation of microtubules and actin microfilaments. Transmembrane protein receptors of the integrin class found in focal adhesions on the cell surface orchestrate the assembly and disruption of cytoskeletal structure and regulation. Upregulation of integrins and extensive assembly of cytoskeletal proteins results in various pathological conditions such as cancer resistance, insulin secretion resistance and neurodegenerative diseases (Alzheimer's disease, Parkinson's disease, Huntington's disease and amyotrophic lateral sclerosis (ALS) (Wilson D M et al. Cell. 2023 Feb. 16; 186 (4): 693-714).

### Neurodegenerative Diseases

Neurodegenerative diseases are characterized by proliferation of beta-amyloid peptide (AB) plaques in neurons. The Worldwide incidence of Alzheimer's disease is 55 million annually, and the worldwide incidence of Parkinson's disease is 8.5 million annually. Huntington's disease affects approximately 1 in 10,000 people worldwide, with about 30,000 people in the United States Neurodegenerative diseases are characterized by proliferation of beta-amyloid peptide (AB) plaques in neurons. Neurodegenerative diseases, primarily Alzheimer's disease and Parkinson's disease, are chronic conditions that permanently damage and destroy a person's nervous system over time, especially their brain. These neurodegenerative diseases are characterized by the proliferation of beta-amyloid peptide (AB) plaques in neurons. Cytoskeleton remodeling is positively involved in neurodegenerative diseases. Published studies have reported that the proliferation of beta-amyloid peptide (AB) plaques results from processing of amyloid precursor protein (APP) and F-actin accumulation in neuronal cells this play major roles in the development of Alzheimer's disease (O'Brien R J, et al. Annu Rev Neurosci. 2011; 34:185-204: Fulga T A, et al. Nat Cell Biol. 2007 February; 9 (2): 139-48). Huntington disease (HD) is a genetic neurodegenerative disease disorder that causes the progressive degeneration of nerve cells in the brain regulated by the dysfunction of cellular morphology regulated by huntingtin and cytoskeletal architecture of the cell (Wu, J “The Roles of Cytoskeleton in Huntington's Disease” in proceedings of the 1st Conference on health Data and Intelligent Healthcare pp 572-576 (2022)). These neurodegenerative diseases are incurable.

### Cardiovascular Disease

Atherosclerosis represents the major cause of cardiovascular disease. Atherosclerosis is a chronic inflammatory disease of blood vessels and the brain. The Worldwide incidence of atherosclerosis represents the majority of the 17.3 million cases of cardiovascular disease annually. Cytoskeleton remodeling is positively involved in the development of atherosclerosis. Published studies have reported that actin cytoskeletal organization and the focal adhesion cell-surface receptor integrin signaling drives inflammation is atherosclerosis (Yurdagul A Jr, et al. Arterioscler Thromb Vasc Biol. 2014 July; 34 (7): 1362-73; Rezende L, et al. Biochim Biophys Acta Biomembr. 2022 Sep. 1; 1864 (9): 183951). Atherosclerosis is incurable.

### Insulin Resistance in Diabetics

The worldwide incidence of diabetes is about 422 million people, and the worldwide number of patients deaths is 6-7 million people annually. Impaired glucose uptake and glucose-stimulated insulin secretion (GSIS) are major clinical problems for patients with diabetes. Actin cytoskeleton. remodeling is positively involved in glucose-stimulated pancreatic β cell insulin secretion (GSIS) (Rondas D, et al. J Biol Chem. 2012 Jan. 20; 287 (4): 2423-36; Rondas D, et al. Diabetes. 2011 April; 60 (4): 1146-57.). Enhancing or disrupting actin polymerization in the cytoskeleton markedly enhances glucose-stimulated insulin secretion (Polino A J, et al. J Biol Chem. 2023 November; 299 (11): 105334; Kalwat M A, et al. Exp Mol Med. 2013 Aug. 23; 45 (8): e37; Arous C, et al. Am J Physiol Endocrinol Metab. 2015 Oct. 1; 309 (7): E611-20]. Published studies have reported that the focal adhesion cell-surface receptor integrin and its interaction with the extracellular matrix and the actin cytoskeleton regulates glucose metabolism in skeletal muscle. (Muldoon J T, et al. 1985. Am J Public Health. 1987 August; 77 (8): 1006-8.). Studies have further reported that activated integrins regulate insulin action [MID: 33359386], that integrins directly bind to insulin-like growth factor (IGF-1) and mediate insulin signaling (Saegusa J, et al. J Biol Chem. 2009 Sep. 4; 284 (36): 24106-14; Fujita M, et al. J Biol Chem. 2012 Apr. 6; 287 (15): 12491-500; Takada Y, et al. Cytokine Growth Factor Rev. 2017 April; 34:67-72). Insulin resistance in diabetes is incurable.

### Objects of the Invention

It is an object of the present invention to provide compounds useful for treating non-cancer patients having intractable disease caused by aberrant α5β1 focal adhesion dynamics and cytoskeletal organization.

It is a further object of the present invention to provide compounds useful for reducing disease progression in non-cancer patients with neurodegenerative disease, atherosclerosis and diabetes.

It is a further object of the present invention to provide compounds useful for reducing disease progression non-cancer patients with Alzheimer's disease, Parkinson's disease, Huntington's disease, atherosclerosis and insulin-resistant diabetes.

It is a further object of the present invention for reducing disease progression non-cancer patients having intractable disease caused by aberrant α5β1 focal adhesion dynamics and cytoskeletal organization with compounds that have a low risk of causing ventricular arrhythmias, reduced dopaminergic side effects, reduced hERG binding and reduced QTc interval prolongation.

### BRIEF SUMMARY OF THE INVENTION

The present invention relates to compounds and pharmaceutical compositions for treating a subject having neurodegenerative disease, atherosclerosis or diabetes in need of therapy thereof comprising administering to the subject a compound in an amount effective in inhibiting disease progression, said compound having the formula:

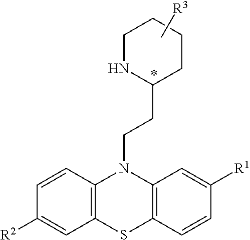

where R¹ is hydrogen, halogen, trifluoromethyl or an alkylthio group, R² is hydrogen, alkoxy or a branched or a straight chain acyclic alkyl group containing from one to about five carbon atoms, and R³ is hydrogen, lower alkyl or lower alkoxy, and a pharmaceutically acceptable carrier, pharmaceutically acceptable salt or a controlled release carrier. The compounds of the invention advantageously control disease progression and have low cardiotoxicity risk.

### DETAILED DESCRIPTION OF THE INVENTION

In accordance with the present invention, provided are compounds having the formula:

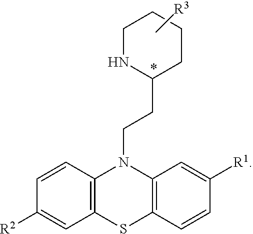

where R¹ is hydrogen, halogen, trifluoromethyl or an alkylthio group, R² is hydrogen, alkoxy or a branched or a straight chain acyclic alkyl group containing from one to about five carbon atoms, and R⁵ is hydrogen, lower alkyl or lower alkoxy, * is a chiral center and a pharmaceutically acceptable carrier, pharmaceutically acceptable salt, enantiomer or a controlled release carrier.

In accordance with the present invention, R¹ is hydrogen, halogen, trifluoromethyl, sulfhydryl or an alkylthio group. In a preferred embodiment of the present invention, R¹ is trifluoromethyl or an alkylthio group. In accordance with the present invention, the alkylthio group is a sulfone wherein the sulfonyl group is connected to an alkyl having from 1 to about three carbon atoms and to a ring carbon. In a preferred embodiment of the present invention, the alkylthio is thiomethyl having the formula: —S(CH₃).

In accordance with the present invention, lower alkyls are those alkyls containing a branched or straight chain acyclic alkyl group containing from one to five carbon atoms and include methyl, ethyl, propyl, isopropyl, butyl, isobutyl and pentyl. In accordance with a preferred embodiment of the present invention, lower alkyls are those containing from 1 to about three carbon atoms. In accordance with the present invention, lower alkoxy groups include methoxy, ethoxy, propoxy such as n-propoxy, butoxy such as n-butoxy and t-butoxy, and pentoxy such as n-pentoxy. In accordance with the present invention, halogens are preferably fluorine and chlorine.

In accordance with the present invention, R² is hydrogen, alkoxy or lower alkyl including but not limited to methyl, ethyl, propyl or butyl. In accordance with a preferred embodiment of the present invention, R² is hydrogen, alkoxy or a branched or a straight chain acyclic alkyl group containing from one to about three carbon atoms, including but not limited to methyl, ethyl, and propyl. In one embodiment of the present invention, R² is preferably hydrogen. In another embodiment of the present invention R² is an alkoxy or a branched or a straight chain acyclic alkyl group containing from one to about five carbon atoms. In accordance with a preferred embodiment of the present invention, R² is propyl.

In accordance with the present invention, R³ is hydrogen, a lower alkyl or a lower alkoxy. In accordance with one embodiment of this invention, R³ is methyl or ethyl. In a more preferred embodiment of the present invention, R³ is hydrogen.

In accordance with a preferred embodiment of the present invention, provided are enantiomeric compounds having the formula:

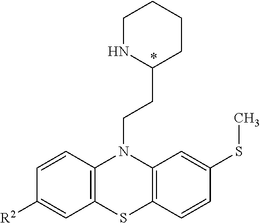

In accordance with the present invention purified enantiomers are those that are not a racemate or are non-superimposable mirror images of each other with a chiral center. In accordance with this invention, the optical rotation for the starting material, [2R)-piperidin-2-yl] acetic acid, for the R isomer was [α]25D=+0.1 (c=0.1, MeOH). Likewise, in accordance with the present invention, the optical rotation for the starting material, [(2S)-piperidin-2-yl] acetic acid, for the S isomer was [α]25D=+0.1 (c=0.1, MeOH).

The compounds of the present invention advantageously control therapy-resistant diseases in non-cancer patients. U.S. Pat. No. 9,198,916 describes similar compounds for controlling tumor growth in cancer patients; however, this patent does not describe the treatment of non-cancer patients. The teachings of U.S. Pat. No. 9,198,916 are hereby incorporated by references.

The compounds of the present invention can be administered as monotherapy or in combination with other therapies. The compounds of the present invention can be administered orally or by parenteral administration. The presently claimed compounds provide an unexpected advantage over similar compounds in the prior art, particularly thioridazine. Thioridazine's cardiotoxicity in patients is believed to be caused by the excessive prolongation of QT interval (Hartigan-Go et al. Clinical Pharmacology & Therapeutics 60:543-553 (1996), and a direct result of its high hERG liability. Surprisingly and unexpectedly, the compounds of the present invention have an advantage over previously used phenothiazine compounds like thioridazine because they have a low hERG liability.

In accordance with another embodiment of the present invention, the compounds of the present invention are administered orally. In this embodiment, the presently claimed compounds provide an additional unexpected advantage over similar compounds in the prior art, particularly thioridazine. Orally administered phenothiazines like thioridazine, for example, have demonstrated a propensity toward oxidation by cytochrome P450 2D6 enzymes to 7-hydroxy quinoneimine metabolites that have their oxidized (hydroxyl) group in the para-position to the ring nitrogen in the phenothiazine structure. Without being held to a particular theory or mechanism, we believe that the highly reactive quinoneimine toxicophores that are produced when phenothiazines-based drugs are administered orally are responsible in part for the proarrhythmic activity that has been reported to cause severe life-threatening arrhythmias (torsade de pointes) and sudden death. Compounds in which the highly reactive quinoneimine toxicophores are not produced provide a significant advantage over phenothiazines such as thioridazine.

The preparation and use of compounds and prodrugs in accordance with the present invention will be readily apparent to those skilled in the art and the well-known and well-documented procedures for substituted phenothiazine-based drugs in the scientific and patent literature. The following United States patents provide illustrations on the synthesis of the phenothiazine analogs, and are hereby incorporated by reference in their entirety, together with the patents cited therein: U.S. Pat. Nos. 2,905,590; 3,310,553; 4,107,430; 4,042,695; 3,951,961; 6,407,231; 5,503,759; 3,305,547; 9,198,916 and 8,088,918.

Synthesis and preparation of enantiomeric compounds in accordance with the present invention can be prepared by those of ordinary skill in the art without undue experimentation as taught in U.S. Pat. No. 6,020,506, which is specifically incorporated herein by reference for the purpose of describing and enabling the preparation of compositions comprising enantiomerically pure compounds. Methods of purification will be well known to those of ordinary skill and may include, e.g., dissolution of the mixture in a solvent and recrystallization. Column chromatography may be used to resolve a phenothiazine racemate into its two enantiomers. Racemates may also be separated using chromatographic separation, such as gas chromatography (GC) or high-performance liquid chromatography (HPLC), such as used in the resolution of promethazine, ethopropazine, trimeprazine and trimipramine enantiomers (Ponder G W, Stewart J T. J Pharm Biomed Anal. 1995 August; 13 (9): 1161-6.) Capillary electrophoresis (CE) may also be employed (Wang et al., 2001). These compounds can also be easily prepared by the synthetic scheme or that described by Daniel et al. Pol. J. Pharmacol. 49 (6): 439-452 (1997); Daniel et al. Exp. Toxicol. Pathol. 51 (4-5): 309-314 (1999); Daniel et al. British Journal of Pharmacology 131:287-295 (2000). Starting materials for compounds where R² is other than hydrogen, for example, include lower alkyls such as 3-methyl-10H-phenothiazine (CAS 3939-47-7), 3-ethyl-10H-phenothiazine (CAS 54027-87-1), 3-propyl-10H-phenothiazine (CAS 92-33-1), 3-butyl-10H-phenothiazine (CID 70288115), 3-propan-2yl-10H-phenothiazine (CID 70290282), 3-(1,1 dimethylethyl)-10H-phenothiazine (CAS 7678-79-7) and alkoxy such as 3-methoxy-10H-phenothiazine (CAS 1771-19-3).

Compounds of the present invention can be prepared as prodrugs that avoid biotransformation by cytochrome P450 2D6 to quinoneimine metabolites. The preparation of these prodrugs is described in U.S. Pat. No. 8,088,918 which is hereby incorporated by reference.

In accordance with another preferred embodiment of the present invention, compounds are provided having the formula:

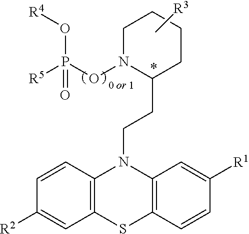

where R¹ is hydrogen, halogen, trifluoromethyl or an alkylthio group, R² is hydrogen, alkoxy or a branched or a straight chain acyclic alkyl group containing from about one to about five carbon atoms, and R³ is hydrogen, lower alkyl or lower alkoxy, and a pharmaceutically acceptable carrier, salt or a controlled release carrier.

Determining the dose and the amounts of compounds effective in treating a subject having a disease and in need of therapy in accordance with the present invention will be readily apparent to those skilled in the art. The following examples include the treatments of major diseases in accordance with the present invention, but they are not intended to be limiting. In accordance with one aspect of the present invention, a method for treating a subject having Alzheimer's disease comprises administering to the subject a compound in an amount effective in modulating progression of Alzheimer's disease. In accordance with another aspect of the present invention, a method for treating a subject having Parkinson's disease, comprises administering to the subject a compound in an amount effective in modulating progression of Parkinson's disease. In accordance with another aspect of the present invention, a method for treating a subject having Huntington's disease, comprises administering to the subject a compound in an amount effective in modulating progression of Huntington's disease. In accordance with another aspect of the present invention, a method for treating a subject having atherosclerosis comprises administering to the subject a compound in an amount effective in modulating progression of atherosclerosis. In accordance with another aspect of the present invention, a method for treating a subject having insulin-resistant diabetes comprises administering to the subject a compound in an amount effective in modulating progression of insulin-resistant diabetes.

The compounds of the present invention can be administered in admixture with a suitable pharmaceutical excipient, diluent or carrier selected with regard to the intended route of administration and standard pharmaceutical practice. The term “carrier” refers to diluents, excipients and the like for use in preparing admixtures of a pharmaceutical composition. For example, the compounds of the present invention can be administered orally in the form of tablets, capsules, multi-particulates, elixirs, solutions or suspensions, which may contain flavoring or coloring agents, either for immediate-, delayed-, modified-, sustained-, pulsed- or controlled-release applications. Suitable formulations of the compounds of the present invention may be in coated or uncoated form, as desired. Prodrugs in accordance with the present invention may be pH-labile and require delayed-release formulations to protect the prodrug from hydrolysis in the stomach. Preferably these delayed-release formulations contain enteric coatings. Pharmaceutically acceptable carriers include but are not limited to sterile water, saline, buffered saline, dextrose solution, preferably such physiologically compatible buffers as Hank's or Ringer's solution, physiological saline, a mixture consisting of saline and glucose, and heparinized sodium-citrate-citric acid-dextrose solution and the like. As used herein, the term “pharmaceutically acceptable” means approved by a regulatory agency of the Federal or a state government or listed in the U.S. Pharmacopeia or other generally recognized pharmacopeia for use in animals, and more particularly in humans.

Unless otherwise specifically identified or claimed for preferred embodiments, the following general definitions are used in accordance with the present invention. In accordance with the present invention, the term “aberrant α5β1 focal adhesion dynamics and cytoskeletal organization” refers to the regulation of the actin-cytoskeletal architecture by the α5β1 integrin cell surface receptor. In accordance with the present invention, the term “to target” or “to targeted” refers to the recognition of a target and delivery of a drug to that target; however, no internalization of the drug is inferred. In accordance with the present invention, the term “selectively target” refers to selective preference of one cell type over another. In accordance with the present invention, the term “modulate” refers to a change in the parameter measured, such that modulate can mean either an increase or decrease. In a preferred embodiment of the present invention, “modulate” refers to a reduction in disease progression.

The present invention has been described in detail using specific examples to illustrate the preferred embodiments of the invention; however, it will be obvious to those skilled in the art that various modifications thereto can be made without departing from the spirit and scope thereof.

### EXAMPLES OF THE INVENTION

### Example 1:2-(methylsulfanyl)-10-{2-[(2R)-piperidin-2-yl]ethyl}-10Hphenothiazine dihydrochloride

2-(methylsulfanyl)-10-{2-[(2R)-piperidin-2-yl]ethyl}-10Hphenothiazine dihydrochloride was prepared using [(2R)-piperidin-2-yl]acetic acid as the purified enantiomer starting material. The optical rotation of the starting material was [α]25D=+0.1 (c=0.1, MeOH). 2-(methylsulfanyl)-10-{2-[(2R)-piperidin-2-yl]ethyl}-10Hphenothiazine dihydrochloride was produced in 95% yield by 1H NMR analysis and 95.2% by LCMS analysis and appeared as a green crystalline powder. The elemental analysis for 2-(methylsulfanyl)-10-{2-[(2R)-piperidin-2-yl]ethyl}-10Hphenothiazine dihydrochloride is C20H26Cl2N2S2, the formula weight is 429.4698, and the moisture content using the “Karl Fisher” method is 1.40% (by weight).

### Example 2:2-(methylsulfanyl)-10-{2-[(2S)-piperidin-2-yl]ethyl}-10H-phenothiazine dihydrochloride

2-(methylsulfanyl)-10-[2-[(2S)-piperidin-2-yl]ethyl]-10H-phenothiazine dihydrochloride was prepared using [(2S)-piperidin-2-yl]acetic acid as the purified enantiomer starting material. The optical rotation of the starting material was [α]25D=−0.4 (c=0.1, MeOH). 2-(methylsulfanyl)-10-{2-[(2S)-piperidin-2-yl]ethyl}-10H-phenothiazine dihydrochloride was produced in 95% yield by 1H NMR analysis and 95% by LCMS analysis and appeared as a green crystalline powder. The elemental analysis for 2-(methylsulfanyl)-10-{2-[(2S)-piperidin-2-yl]ethyl}-10Hphenothiazine dihydrochloride is C20H26Cl2N2S2, the formula weight is 429.4698, and the moisture content using the “Karl Fisher” method is 1.29% (by weight).

What is claimed is:

1. A method for treating for treating non-cancer patients having an intractable disease caused by aberrant α5β1 focal adhesion dynamics and cytoskeletal organization in need of therapy thereof comprising administering to the subject a compound in an amount effective in reducing progression of said disease, said compound having the formula: 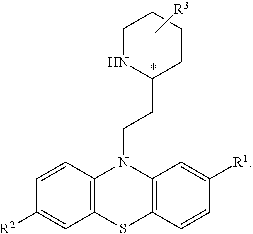
where R¹ is hydrogen, trifluoromethyl or an alkylthio group, R² is hydrogen, alkoxy or a branched or a straight chain acyclic alkyl group containing from one to about five carbon atoms, and R³ is hydrogen, lower alkyl or lower alkoxy, and a pharmaceutically acceptable carrier, salt, enantiomer or a controlled release carrier.

2. The method of claim 1 wherein R¹ is an alkylthio group, R² is hydrogen or a straight chain alkyl containing from one to about five carbon atoms, R³ is hydrogen, and the enantiomer is the (+) enantiomer.

3. The method of claim 1 wherein R¹ is an alkylthio group, R² is hydrogen or a straight chain alkyl containing from one to about five carbon atoms, R³ is hydrogen, and the enantiomer is the (−) enantiomer.

4. The method of claim 1 wherein said compound has the formula 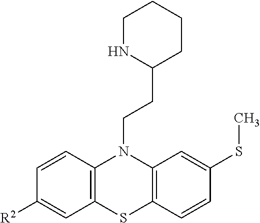
where R² is alkoxy or lower alkyl having from one to about three carbon atoms.

5. The method of claim 4 wherein said compound has the formula 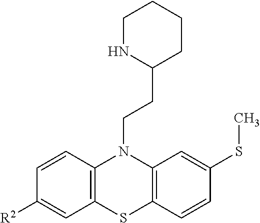
where R² is straight chain lower alkyl having from one to about three carbon atoms.

6. A method for treating non-cancer patients having a neurological disease in need of therapy thereof comprising administering to the subject a compound in an amount effective in reducing progression of said neurological disease, said compound having the formula: 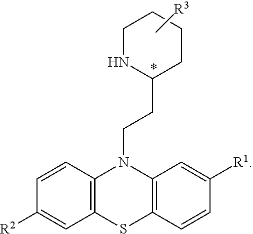
where R¹ is hydrogen, trifluoromethyl or an alkylthio group, R² is hydrogen, alkoxy or a branched or a straight chain acyclic alkyl group containing from one to about five carbon atoms, and R³ is hydrogen, lower alkyl or lower alkoxy, and a pharmaceutically acceptable carrier, salt, enantiomer or a controlled release carrier.

7. The method of claim 6 wherein said neurological disease is Alzheimer's disease, Parkinson's disease, or Huntington's disease.

8. The method of claim 7 wherein said compound has the formula 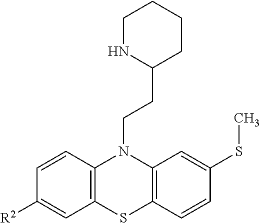
where R² is alkoxy or lower alkyl having from one to about three carbon atoms.

9. The method of claim 7 wherein said compound has the formula 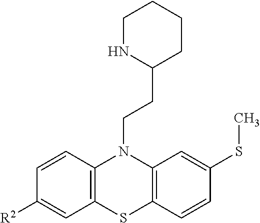
where R² is straight chain lower alkyl having from one to about three carbon atoms.

10. A method for treating non-cancer patients having atherosclerosis in need of therapy thereof comprising administering to the subject a compound in an amount effective in reducing progression of said atherosclerosis, said compound having the formula 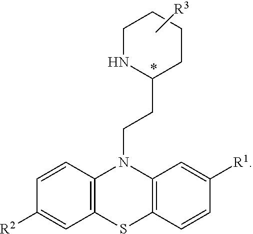
where R¹ is hydrogen, trifluoromethyl or an alkylthio group, R² is hydrogen, alkoxy or a branched or a straight chain acyclic alkyl group containing from one to about five carbon atoms, and R³ is hydrogen, lower alkyl or lower alkoxy, and a pharmaceutically acceptable carrier, salt, enantiomer or a controlled release carrier.

11. The method of claim 10 wherein said compound has the formula 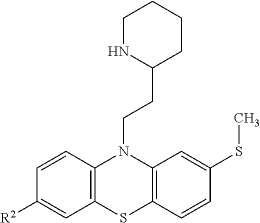
where R² is alkoxy or lower alkyl having from one to about three carbon atoms.

12. The method of claim 10 wherein said compound has the formula 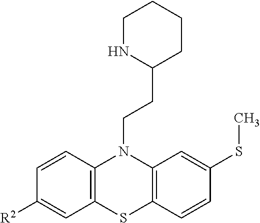
where R² is straight chain lower alkyl having from one to about three carbon atoms.

13. A method for treating non-cancer patients having insulin-resistance in need of therapy thereof comprising administering to the subject a compound in an amount effective in reducing progression of said insulin-resistance, said compound having the formula 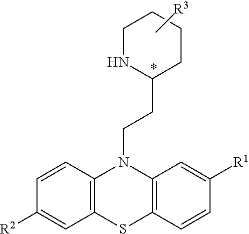
where R¹ is hydrogen, trifluoromethyl or an alkylthio group, R² is hydrogen, alkoxy or a branched or a straight chain acyclic alkyl group containing from one to about five carbon atoms, and R³ is hydrogen, lower alkyl or lower alkoxy, and a pharmaceutically acceptable carrier, salt, enantiomer or a controlled release carrier.

14. The method of claim 13 wherein said compound has the formula 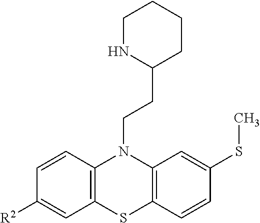
where R² is alkoxy or lower alkyl having from one to about three carbon atoms.

15. The method of claim 13 wherein said compound has the formula 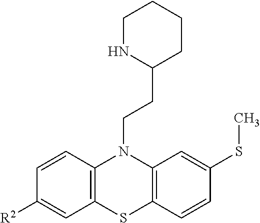
where R² is straight chain lower alkyl having from one to about three carbon atoms.
